(19) United States
(12) Patent Application Publication
(10) Pub. No.: US 2016/0166253 A1
(43) Pub. Date: Jun. 16, 2016

(54) STAPLING DEVICE WITH DISTALLY LOCATED HYDRAULIC DRIVE- ROTARY OPERATED SYSTEM AND METHOD

(71) Applicant: Cardica, Inc., Redwood City, CA (US)

(72) Inventor: Bryan D. Knodel, Flagstaff, AZ (US)

(21) Appl. No.: 14/793,641

(22) Filed: Jul. 7, 2015

Related U.S. Application Data
(60) Provisional application No. 62/091,251, filed on Dec. 12, 2014.

(51) Int. Cl. A61B 17/072

(52) U.S. Cl. CPC A61B 17/07207

(57) ABSTRACT

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a control member to close the jaw assembly to clamp, staple, and/or cut the target tissue. The end-effector also includes a rotary hydraulic drive system to provide direct drive power next the distal portion of the end-effector to drive the deployment operations, such as deploying staples and cutting tissue.

### FIELD OF THE INVENTION

This Nonprovisional Application claims priority to Provisional Application No. 62/091,251 filed Dec. 12, 2014.

The present invention relates generally to surgical devices, and more particularly to surgical stapling or clip applying systems.

### BACKGROUND

Surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient. A typical stapling device holds a disposable single-use cartridge with typically two or more rows of staples, and includes an anvil to oppose the staples as the staples are deployed from the cartridge. During operations, the surgeon inserts the stapling device through an opening in the body (usually using a trocar), orients the end of the stapling device around the tissue to be transected, and compresses the anvil and cartridge together to clamp that tissue. Then, a row or rows of staples are deployed on either side of the transection line, and a blade is advanced along the transection line to cut and/or divide the tissue.

During actuation of the endocutter, the stapling device fires all of the staples in the single-use disposable cartridge. In order to deploy more staples, the endocutter must be moved away from the surgical site and removed from within the patient. The spent cartridge is removed from the endocutter and replaced by a new cartridge. The endocutter is then reinserted into the patient for further staple deployment.

Accordingly, it would be desirable to miniaturize the components within the end-effector of the stapling device to allow for greater operability within a small space and ease of operation.

### SUMMARY OF THE INVENTION

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a clamp control member to close the jaw assembly on the target tissue. The stapling device includes a rotary hydraulic drive system within the end-effector to provide direct driving of a deployment assembly member to deploy staples.

A surgical stapling device is configured for use in open and/or laparoscopic surgical procedures. The device includes a handle assembly, a shaft assembly coupled to the handle assembly, and an end-effector coupled to the shaft assembly. The end-effector comprises of a jaw assembly configured to clamp, staple, and/or cut a target tissue. The handle assembly comprises of a trigger member that can activate a control member to close the jaw assembly to clamp, staple, and/or cut the target tissue. The end-effector also includes a rotary hydraulic drive system to provide direct drive power next the distal portion of the end-effector to drive the deployment operations, such as deploying staples and cutting tissue.

The stapling device as described in this disclosure comprises of a shaft member coupled to the end-effector, wherein the shaft member includes a flexible segment to allow articulation of the end-effector. In addition, hydraulic supply lines are routed from within the shaft member to the end-effector to operate the rotary hydraulic drive system within the end-effector.

The stapling device as described in this disclosure, wherein in some embodiments the rotary hydraulic drive system is disposed within the lower jaw member of the end-effector.

The stapling device as described in this disclosure, wherein in some embodiments the rotary hydraulic drive system includes a drive power member to operate a drive control member that advances or retracts the deployment assembly member to execute deployment operations or to execute reset operations.

The stapling device as described in this disclosure, wherein in some embodiments the drive power member is a drive gear or a drive pulley and the drive control member is cable.

The stapling device as described in this disclosure, wherein in some embodiments the drive power member is a drive gear and the drive control member is screw drive rod.

The stapling device as described in this disclosure, wherein in some embodiments the hydraulic drive system includes a pair of drive power members, wherein each of the pair of drive power members operates a respective drive control member that advances or retracts a corresponding sub-assembly member of the deployment assembly member to execute deployment operations or to execute reset operations.

The stapling device as described in this disclosure, in some embodiments the corresponding sub-assembly member comprises of a wedge member configured to deploy staples.

The stapling device as described in this disclosure, in some embodiments, the hydraulic drive system can selectively drive one or both of the pair of drive power members, such that each of the pair of drive power members can be operated separately or independently.

The stapling device as described in this disclosure, in some embodiments, each of the pair of the drive power members is a drive gear and the respective drive control member is screw drive rod.

### BRIEF DESCRIPTION OF THE DRAWINGS

The present invention will be readily understood by the following detailed description, taken in conjunction with accompanying drawings, illustrating by way of examples of the embodiments of the invention. The figures are merely exemplary and not limiting. The objects and elements in the drawings are not necessarily drawn to scale, proportion, precise orientation or positional relationships; instead, emphasis is focused on illustrating the principles of the invention. Descriptive terms such as “upper,” “lower,” “upward,” “downward”, “forward”, “backward”, and the like are intended for the convenience of the reader and refer to the orientation and/or motion of parts as illustrated and described; they do not necessarily limit the orientation or operation of the features, aspects, or embodiments of the invention. The drawings illustrate the design and utility of various features, aspects, or embodiments of the present invention, in which like element are typically referred to by like reference symbols or numerals. The drawings, however, depict the features, aspects, or embodiments of the invention, and should not be taken as limiting in their scope. With this understanding, the features, aspects, or embodiments of the invention will be described and explained with specificity and details through the use of the accompanying drawings in which:

FIG. 1A illustrates an example of a surgical stapling device, in accordance with features, aspects or embodiments of the present invention.

FIG. 1B illustrates the distal portion of the surgical stapling device which includes an articulation segment that allows an end-effector of the surgical stapling device to articulate, in accordance with features, aspects or embodiments of the present invention.

FIG. 1C illustrates a joint portion or section of the end-effector of the surgical stapling device, wherein the joint portion, as illustrated, is located distally to the articulation segment and proximally to the effector, in accordance with features, aspects or embodiments of the present invention.

FIG. 1D illustrates one example of a hydraulic drive system positioned distally to the joint portion or articulation portion and within the end-effector which allows for direct operation of the control elements of the end-effector, in accordance with features, aspects or embodiments of the present invention.

FIG. 2A illustrates a close-up (side) view of the hydraulic drive system positioned distally to the joint portion or articulation portion and within the end-effector which allows for direct operation of the control elements of the end-effector, in accordance with features, aspects or embodiments of the present invention.

FIG. 2B illustrates a close-up (isometric) view of the hydraulic drive system positioned distally to the joint portion or articulation portion and within the end-effector which allows for direct operation of the control elements of the end-effector, in accordance with features, aspects or embodiments of the present invention.

FIGS. 3A and 3B illustrate the hydraulic drive system that drives the control elements to operate various components in the end-effector of the surgical stapling device, wherein the components include a wedge assembly to deploy staples and a knife member to cut tissue, in accordance with features, aspects or embodiments of the present invention.

FIG. 4A illustrates a close-up (side) view of the hydraulic drive system that drives the control elements to operate various components in the end-effector of the surgical stapling device, wherein the components include a wedge assembly to deploy staples and a knife member to cut tissue, in accordance with features, aspects or embodiments of the present invention.

FIG. 4B illustrates a close-up (isometric) view of the hydraulic drive system that drives the control elements to operate various components in the end-effector of the surgical stapling device, wherein the components include a wedge assembly to deploy staples and a knife member to cut tissue, in accordance with features, aspects or embodiments of the present invention.

FIGS. 5A and 5B illustrate another example of a hydraulic drive system positioned distally to the joint portion or articulation portion (and within the end-effector of the stapling device) which allows for direct operation of the control elements of the end-effector, in accordance with features, aspects or embodiments of the present invention.

FIG. 6A through FIG. 6G illustrate the close-up views of the hydraulic drive system which drives various control elements to operate various components in the end-effector of the surgical stapling device, wherein the components include a wedge assembly to deploy staples and a knife member to cut tissue, in accordance with features, aspects or embodiments of the present invention.

FIGS. 7 illustrates yet another example of a hydraulic drive system positioned distally to the joint portion or articulation portion (and within the end-effector of the stapling device) which allows for direct operation of the control elements of the end-effector, in accordance with features, aspects or embodiments of the present invention.

FIG. 8A through FIG. 8C illustrate the close-up views of the hydraulic drive system which drives various control elements to operate various components in the end-effector of the surgical stapling device, wherein the components include a wedge assembly to deploy staples and a knife member to cut tissue, in accordance with features, aspects or embodiments of the present invention.

### DETAILED DESCRIPTION

In the following detailed description, specific details are set forth in order to provide a thorough understanding of the present invention. However, it will be readily understood by those skilled in the art that the present invention may be practiced without these specific details. Alternatively, some of the well-known parts, components, hardware, methods of operations, and procedures may not be described in detail or elaborated so as to avoid obscuring the present invention; but, nevertheless, they are within the spirit and scope of the present invention.

As mentioned, surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient. A typical stapling device holds a disposable single-use cartridge with several rows of staples, and includes an anvil to oppose the staples as the staples are deployed from the cartridge. During operations, the surgeon inserts the stapling device through an opening in the body (typically using a trocar), orients the end of the stapling device around the tissue to be transected, and compresses the anvil and cartridge together to clamp that tissue. Then, a row or rows of staples are deployed on either side of the transection line, and a blade is advanced along the transection line to divide the tissue.

As can be appreciated, it would be highly desirable to develop and implement miniaturized mechanisms and features that can drive or operate various functions of a surgical stapling device. Towards that end, this disclosure describes a stapling device with a distally located hydraulic drive system in accordance with features, aspects or embodiments of the present invention.

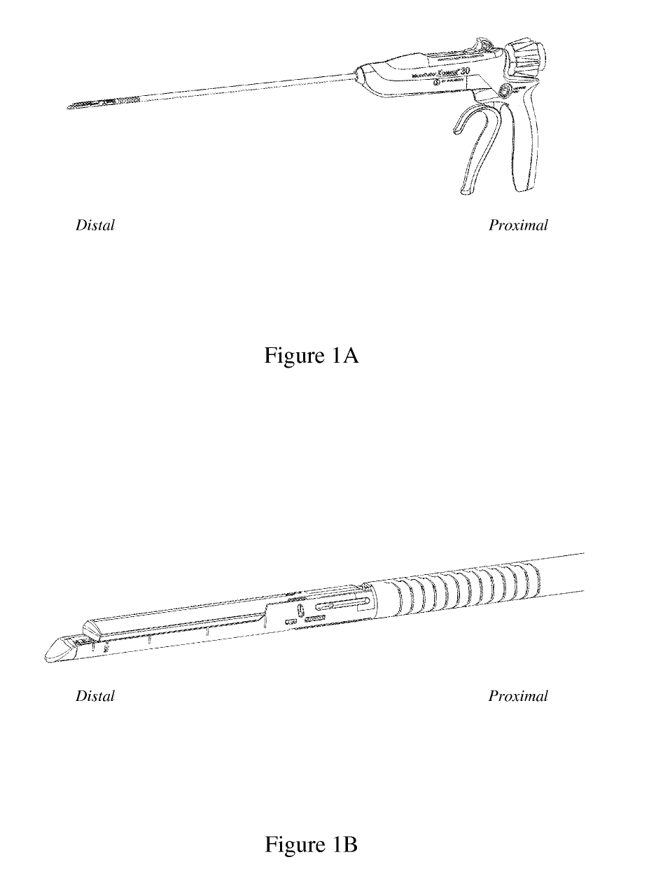

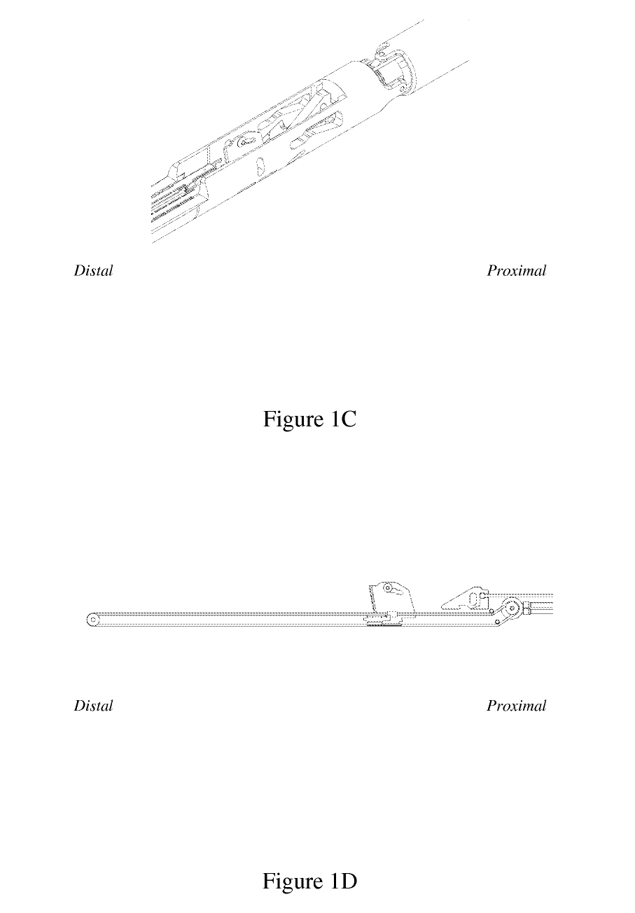

By way of example, FIG. 1A illustrates a surgical stapling device 100 in accordance with features, aspects and embodiments of the present invention. As illustrated, the surgical stapling device 100 includes a body portion 102, a handle portion 104, a trigger member 106, a shaft member 108, and an end-effector 110. FIG. 1B illustrates a close up view of the distal portion of the shaft member 108 along with the end-effector 110. As can be seen, the distal portion of the shaft member 108 may include a flexible segment or flexible region such that the shaft member 108 may be articulated. In some embodiments, the shaft member 108 may include a flexible section (as illustrated), and in some embodiments, the shaft member 108 may be a substantially rigid shaft. Further illustrated in FIG. 1B, the end-effector 110 may include jaw members such as an anvil member 204 and a staple holder channel member 206. The staple holder channel member 206 may be configured to hold a staple cartridge 208. The staple cartridge 208 may include staples and a cutting member for stapling and cutting tissue(s). FIG. 1C illustrates a joint portion or section 210 of the end-effector of the surgical stapling device, wherein the joint portion 210, as illustrated, is located distally to the articulation segment and proximally to the effector. The joint 210 may be a separate component joining, coupling or connecting the shaft member 108 and the end-effector 110. Alternatively, the joint 210 may not be a separate component integral to the shaft member 108 and the end-effector 110. The joint member 210 may be a substantially flexible joint connecting the shaft member 108 and the end-effector 110. FIG. 1D illustrates an example of an embodiment of a rotary hydraulic drive system 140 positioned distally to the joint portion or articulation portion and within the end-effector which allows for direct operation of the control elements of the end-effector, in accordance with features, aspects or embodiments of the present invention. The rotary hydraulic drive system 140 is housed or located distally of the joint portion 210 and near the proximal portion of the end-effector 110. The hydraulic drive system 140 is configured to provide the necessary driving force to operate the deployment operations of the staple device 100. The hydraulic drive system 140 as contained within the end-effector is configured to provide “direct drive input” for deployment operations of the stapling device. Such direct drive input provided by the hydraulic drive system enhances mechanical output advantages. In other words, the features, aspects, and embodiments of the present invention implements a “direct drive” system right near the very tip of the end-effector 110, where deployment operations are executed, to provide increased efficient operating or driving force necessary to performance various deployment operations of the stapling device 100.

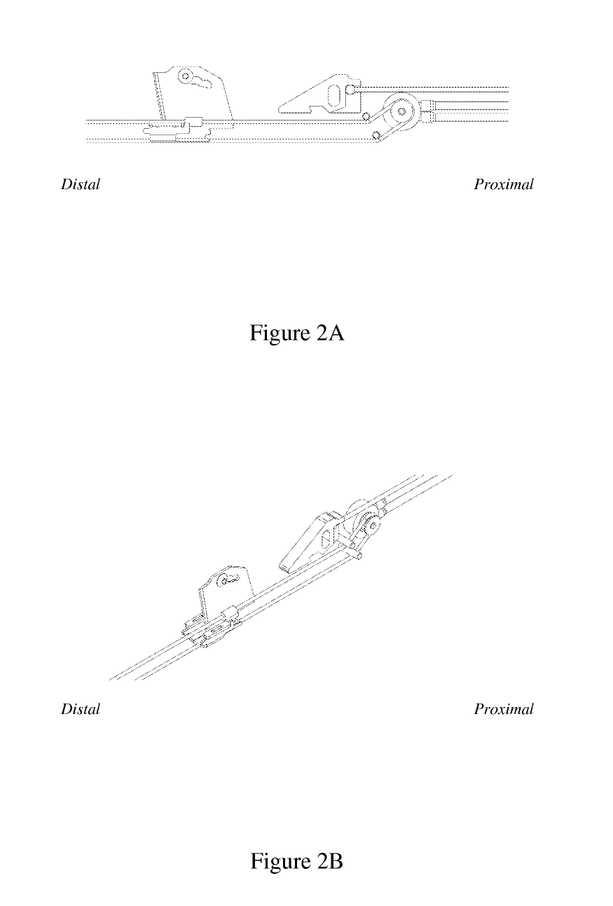

FIG. 2A illustrates a close-up (side) view of the rotary hydraulic drive system 140 positioned distally to the joint portion or articulation portion 210 and within the end-effector 110 which allows for direct operation of the control elements of the end-effector 110. For example, the rotary hydraulic drive system 140 includes input and output hydraulic lines 212 that operate a hydraulic rotary gear or pulley 214 which drives a control element 216, such as a deployment cable. The deployment cable 216 advances or retracts a deployment slide 218. As will be illustrated and discussed in more detail, the deployment slide 218 may be coupled to a staple deployment member and a tissue cutting member (e.g., a staple deployment wedge, a tissue cutting knife, or other similar instruments), such that the deployment cable 216 operatively controls the deployment of staples and cutting of tissue for the stapling device 100. Since the rotary system 140 is located within the end-effector 110, the mechanical advantage of operation is highly efficient as compared to other conventional power supply systems for operating the deployment components of the stapling device. Also illustrated in FIG. 2A is a control member 202 which may be configured to operate the jaw members, e.g., anvil member 204 and staple holder channel 206. FIG. 2B illustrates a close-up (isometric) view of the hydraulic drive system 140 positioned distally to the joint portion or articulation portion 210 and within the end-effector 110 which allows for direct operation of the control elements of the end-effector 110, e.g., staple deployment and tissue cutting members of the stapling device.

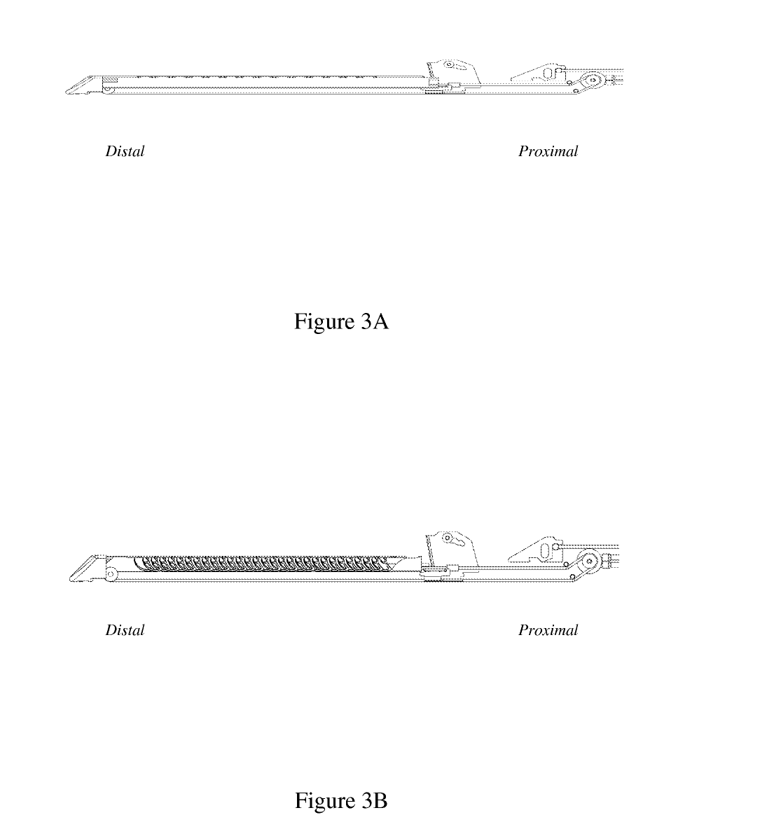

FIG. 3A and FIG. 3B illustrate the rotary hydraulic drive system 140 that drives the control element 216 to operate various components in the end-effector 110 of the surgical stapling device 100, wherein the components may include a deployment slide 218, a wedge assembly 302 to deploy staples 306 in the staple cartridge 208 and a knife member 304 to cut tissue. FIG. 3A illustrates an enclosed staple cartridge 208. In contrast, FIG. 3B illustrates an exposed staple cartridge 208 with the enclosed staples 306 visible. In addition, the wedge assembly 302 is also visible in this exposed view.

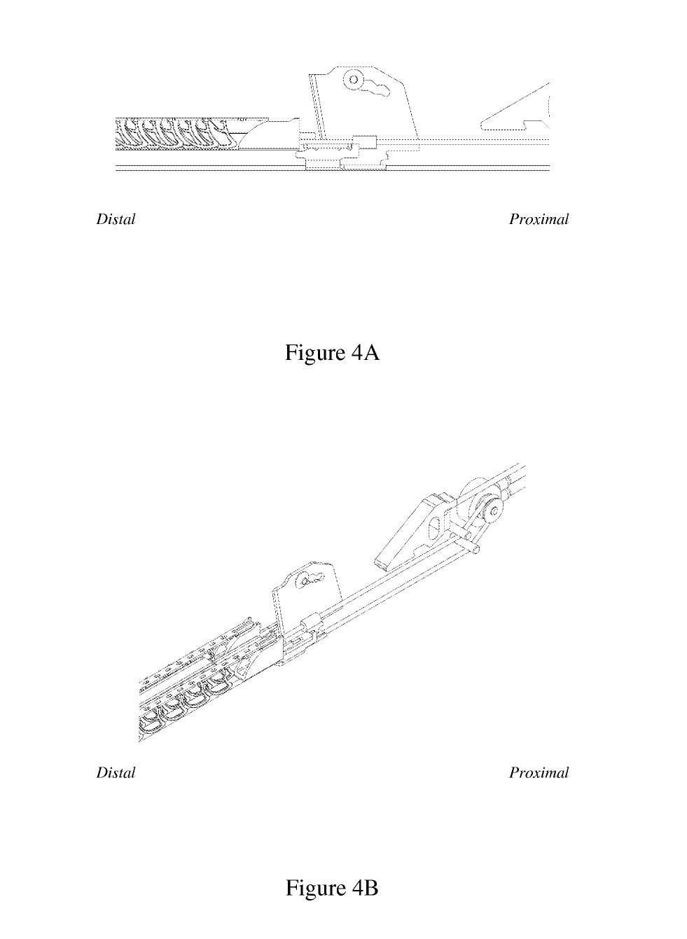

FIG. 4A illustrates a close-up (side) view of the hydraulic drive system 140 that drives the control elements 216 and 218 to operate various components 302 and 304 in the end-effector 110 of the surgical stapling device, wherein the components include a wedge assembly 302 to deploy staples 306 and a knife member 304 to cut tissue. FIG. 4B illustrates a close-up (isometric) view of the hydraulic drive system 140 that drives the control elements (e.g., deployment cable 216 and deployment slide 218) to operate various components (e.g., wedge assembly 302 and cutting member 304) in the end-effector 110 of the surgical stapling device, wherein the components include a wedge assembly 302 to deploy staples 306 and a knife member 304 to cut tissue.

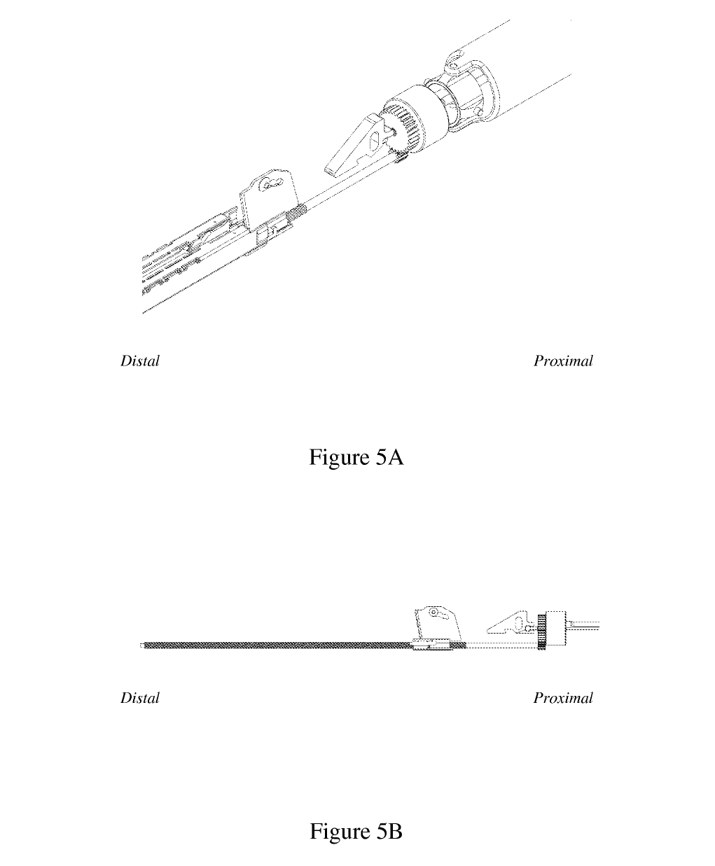

FIG. 5A and FIG. 5B illustrate another example of a hydraulic drive system 500 positioned distally to the joint portion or articulation portion 210 (and within the end-effector 110 of the stapling device) which allows for direct operation of the control elements of the end-effector. As illustrated in 5A, the hydraulic drive system 500 may be comprises of a hydraulic-turbo drive member 502 (e.g., gear, pulley and the like) to operate a drive rod (e.g., a rod screw) 504. The drive rod 504 or rod screw member is coupled to a deployment slide member 506, which is configured to “ride” on the rod screw member 504. When the rod screw member 504 is turned in a first direction by the hydraulic-turbo drive member 502, the deployment slide member 506 may ride forward or advance forward (e.g., distally). When the rod screw member 504 is turned in a second direction by the hydraulic-turbo drive member 502, the deployment slide member 506 may ride backward or retreat backward (e.g., proximally). In a forward deployment mode, the deployment slide member 506 may advance the wedge assembly member 302 and the cutting member 304 to deploy staples 306 in the staple cartridge 208. In a retreat mode, the deployment slide member 506 may retract the wedge assembly member 302 and the cutting member 304 back to a reset position. FIG. 5B illustrates a side view of the hydraulic drive system 500 positioned distally to the joint portion or articulation portion 210 (and within the end-effector 110 of the stapling device).

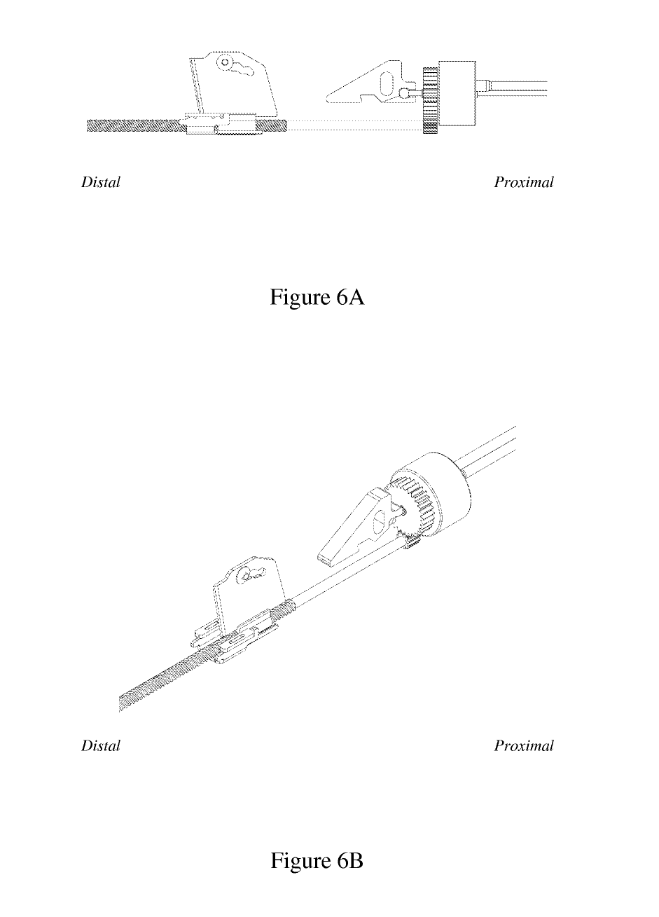

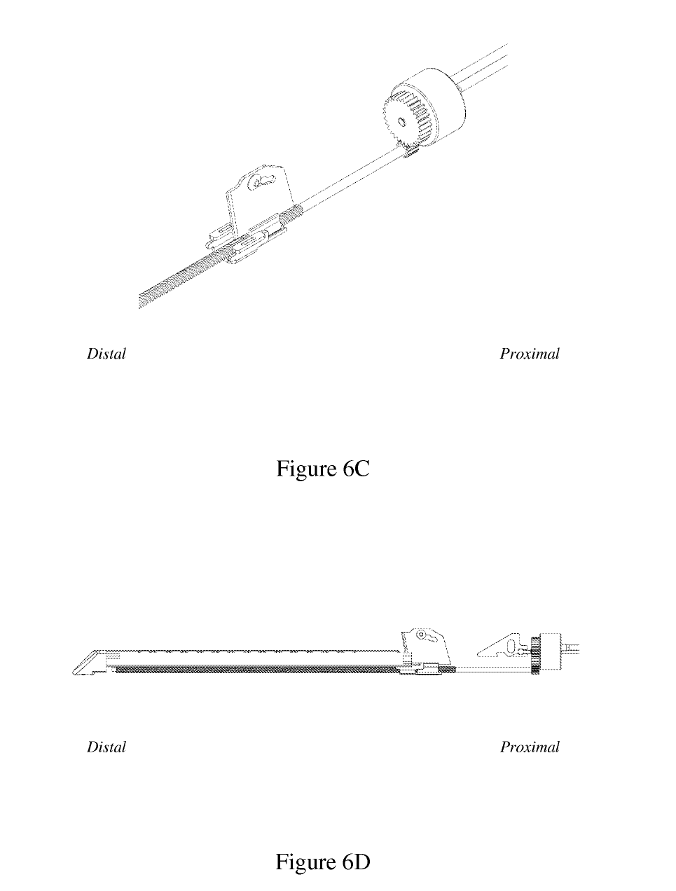

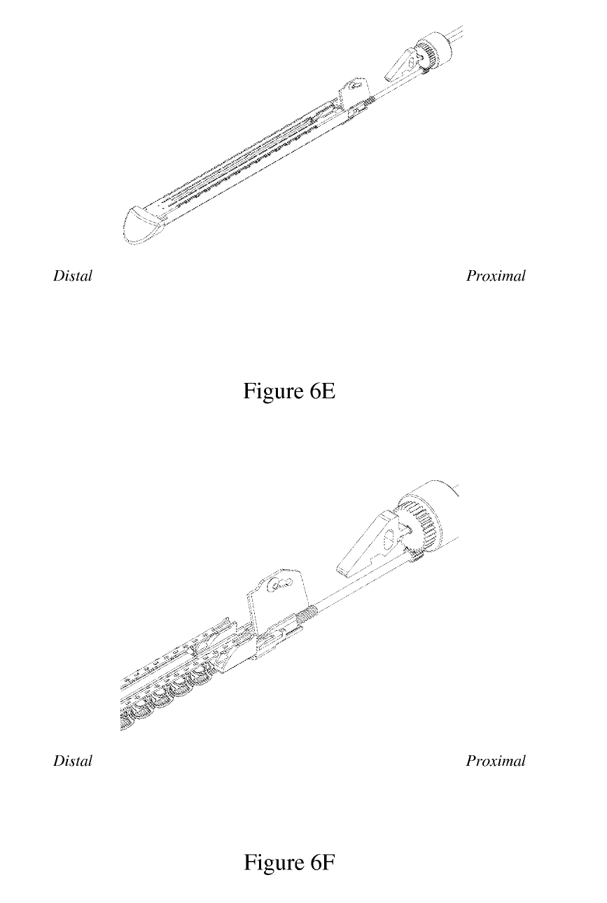

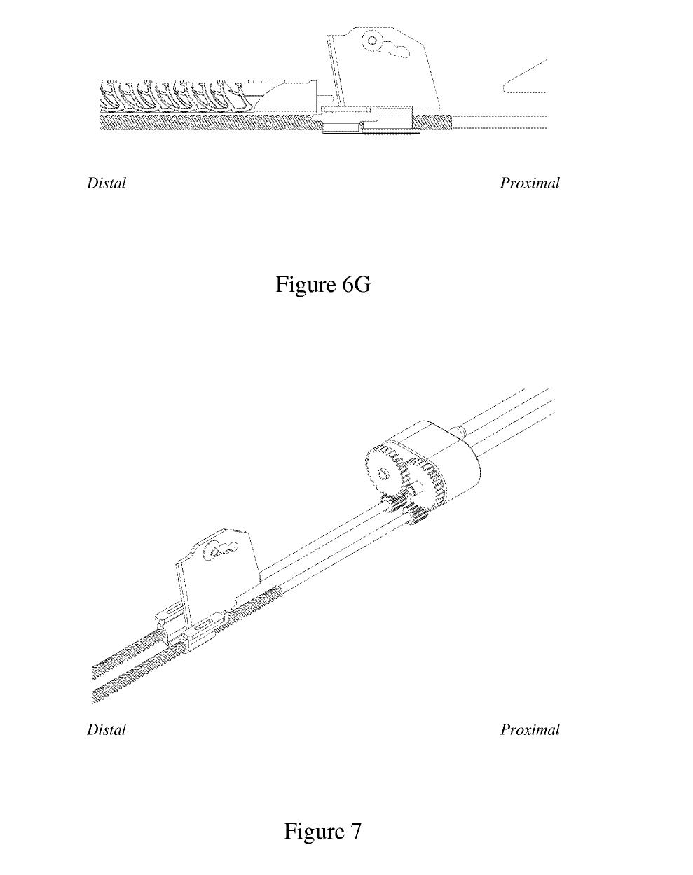

FIG. 6A through FIG. 6G illustrate the close-up views of the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue, in accordance with features, aspects or embodiments of the present invention. For example, FIG. 6A illustrates a close-up side view of the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6B illustrates a close-up perspective view of the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6C illustrates another close-up perspective view of the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6D illustrates a side-view of the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6E illustrates a perspective view with a staple cartridge 208 along with the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6F illustrates a perspective view with an exposed staple cartridge 208 along with the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue. FIG. 6G illustrates a close-up side view with an exposed staple cartridge 208 along with the hydraulic drive system 500 which drives various control elements, e.g., 502, 504 and 506, to operate various components, e.g., 302 and 304 in the end-effector 110 of the surgical stapling device 100, wherein the components include a wedge assembly 302 to deploy staples 306 and a knife member 304 to cut tissue.

FIG. 7 illustrates yet another example of a hydraulic drive system 700 with dual-hydraulic turbo drive members 702 (e.g., gear, pulley and the like) positioned distally to the joint portion or articulation portion 210 (and within the end-effector 110 of the stapling device 100) which allows for direct dual and independent operation of the control elements of the end-effector, in accordance with features, aspects or embodiments of the present invention. As illustrated, the hydraulic drive system 700 operates two hydraulic turbo drive members 702, which each of the turbo drive members 702 may separately and independently operate a deployment control member 504 (such as a direct drive screw rod). Each of the deployment control members 504 may separately and independently operate a deployment slide member 706 to separately and independently advance or retract respective staple deployment wedge member and cutting member to deploy respective staples and cut respective portion or section of target tissue.

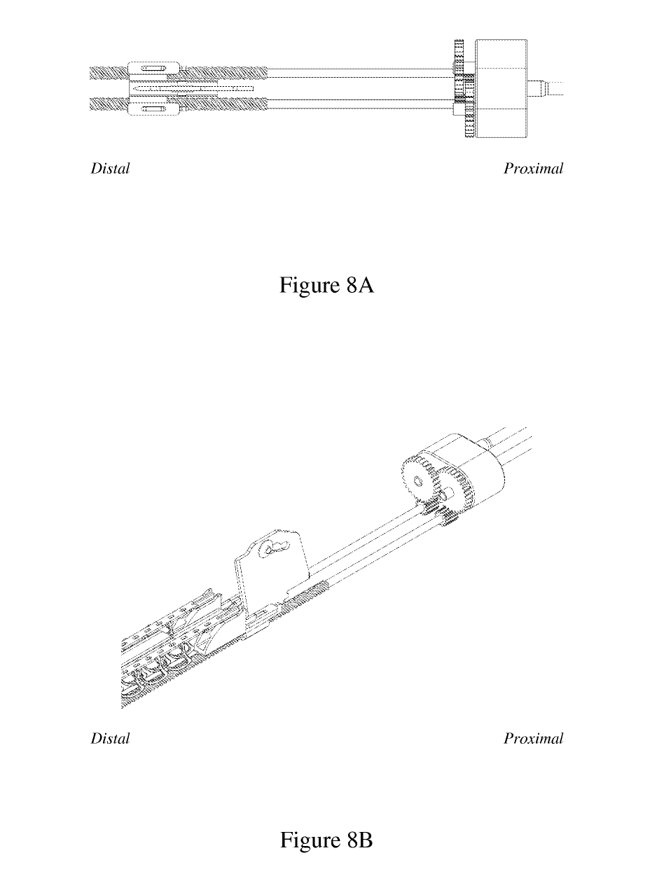

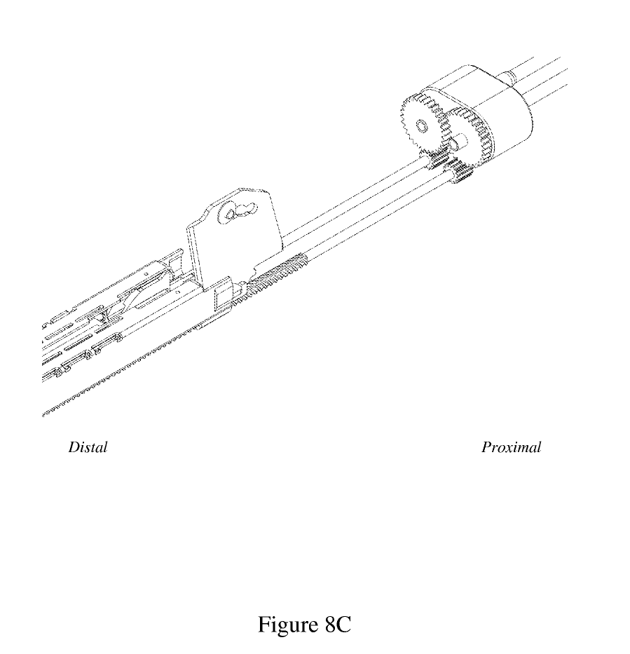

FIG. 8A through FIG. 8C illustrate the close-up views of the hydraulic drive system 700 the dual control elements, e.g., 702, 504 and 706, to operate various components, e.g., 302 and 304, in the end-effector 110 of the surgical stapling device 100, wherein the components include a wedge assembly to deploy staples and a knife member to cut tissue, in accordance with features, aspects or embodiments of the present invention. For example, FIG. 8A illustrates a close-up top view of the hydraulic drive system 700 with dual-hydraulic turbo drive members 702 positioned distally to the joint portion or articulation portion 210 (and within the end-effector 110 of the stapling device 100) which allows for direct dual and independent operation of the control elements of the end-effector. FIG. 8B illustrates a close-up perspective view of the hydraulic drive system 700 with dual-hydraulic turbo drive members 702 positioned distally to the joint portion or articulation portion 210 (and within the end-effector 110 of the stapling device 100), with an exposed view of the staple cartridge 208, which allows for direct dual and independent operation of the control elements of the end-effector. FIG. 8C illustrates a close-up perspective view of the hydraulic drive system 700 with dual-hydraulic turbo drive members 702 positioned distally to the joint portion or articulation portion 210 (and within the end-effector 110 of the stapling device 100), with an unexposed view of the staple cartridge 208, which allows for direct dual and independent operation of the control elements of the end-effector.

Multiple features, aspects, and embodiments of the invention have been disclosed and described by the illustrated figures. Many combinations and permutations of the disclosed invention may be useful in operating a surgical stapling device, and the invention may be configured to support various surgical procedures. One of ordinary skill in the art having the benefit of this disclosure would appreciate that the foregoing illustrated and described features, aspects, and embodiments of the invention may be modified or altered, and it should be understood that the invention generally, as well as the specific features, aspects, and embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure.

Although particular features, aspects, and embodiments of the present invention have been illustrated and described, it should be understood that the above disclosure is not intended to limit the present invention to these features, aspects, and embodiments. It will be obvious to those skilled in the art that various changes and modifications may be made without departing from the spirit and scope of the present invention. Thus, the present invention is intended to cover alternatives, modifications, and equivalents that may fall within the spirit and scope of the following claims and their equivalents.

What is claimed:

1. A stapling device, comprising:
an end-effector with an upper jaw member and a lower jaw member;
a deployment assembly member within the end-effector for deploying staples; and
a rotary hydraulic drive system disposed within the end-effector to provide direct driving of the deployment assembly to deploy the staples.

2. The stapling device of claim 1 further comprising a shaft member coupled to the end-effector, wherein the shaft member includes a flexible segment to allow articulation of the end-effector,
wherein hydraulic supply lines are routed from within the shaft member to the end-effector to operate the rotary hydraulic drive system within the end-effector.

3. The stapling device of claim 1, wherein the rotary hydraulic drive system is disposed within the lower jaw member of the end-effector.

4. The stapling device of claim 1, wherein the hydraulic drive system includes a drive power member to operate a drive control member that advances or retracts the deployment assembly member to execute deployment operations or to execute reset operations.

5. The stapling device of claim 4, wherein the drive power member is a drive gear or a drive pulley, and
wherein the drive control member is cable.

6. The stapling device of claim 4, wherein the drive power member is a drive gear, and
wherein the drive control member is screw drive rod.

7. The stapling device of claim 1, wherein the hydraulic drive system includes a pair of drive power members, wherein each of the pair of drive power members operates a respective drive control member that advances or retracts a corresponding sub-assembly member of the deployment assembly member to execute deployment operations or to execute reset operations.

8. The stapling device of claim 7, wherein the corresponding sub-assembly member comprises of a wedge member configured to deploy staples.

9. The stapling device of claim 7, wherein the hydraulic drive system can selectively drive one or both of the pair of drive power members, such that each of the pair of drive power members can be operated separately or independently.

10. The stapling device of claim 7, wherein each of the pair of the drive power members is a drive gear, and
wherein the respective drive control member is screw drive rod.
